(19) United States
(12) Patent Application Publication
(10) Pub. No.: US 2006/0281951 A1
(43) Pub. Date: Dec. 14, 2006

(54) Commercial ADMA products having reduced salts and odor and the novel process for preparing same

(76) Inventors: John Y. Lee, Baton Rouge, LA (US); Vernon O. Brandt, Baton Rouge, LA (US); Joe P. Sauer, Baton Rouge, LA (US); Paul L. Wiggins, Baton Rouge, LA (US); Wenfeng Kuang, Baton Rouge, LA (US); James R. Adams, Baton Rouge, LA (US); David W. Owens, Baton Rouge, LA (US); Richard D. Glass, Baton Rouge, LA (US); Chau Nguyen, Hattiesbury, MS (US)

Correspondence Address: EDGAR SPIELMAN;ALBEMARLE CORPORATION, 451 FLORIDA BLVD., BATON ROUGE, LA, 70801, US

(21) Appl. No.: 11/447,744

(22) Filed: Jun. 5, 2006

Related U.S. Application Data
(60) Provisional application No. 60/690,320, filed on Jun. 13, 2005.
(60) Provisional application No. 60/704,591, filed on Aug. 1, 2005.

(51) Int. Cl. C07C 211/09

(52) U.S. Cl. 564/511

(57) ABSTRACT

The preset invention relates to low odor ADMA (alkyldimethylamine) products, a process for purifying malodorous impure ADMA products, purified ADMA products, products produced by the purification process, and the use of and the use of the ADMA products as synergists in photopolymerization reactions.

### REFERENCE TO RELATED APPLICATIONS

This application claims priority to U.S. Provisional Patent Application No. 60/690,320 filed Jun. 13, 2005, and U.S. Provisional Patent Application No. 60/704,591 filed Aug. 1, 2005, the disclosure of both herein incorporated by reference in the entirety.

### FIELD OF THE INVENTION

The present invention relates to alkyldimethylamine (ADMA) blends. More particularly, the present invention relates to ADMA products and ADMA blends having reduced malodorous and salt impurities, to a process of removing salts and malodorous impurities in impure ADMA products and blends thereof, and the use of the ADMA products and ADMA blends as synergists in photopolymerization reactions.

### BACKGROUND OF THE INVENTION

Alkyldimethylamines (“ADMA”) products and ADMA blends have many uses such as, for example, ink applications, the manufacture of quarternary ammonium compounds for biocides, textile chemicals, oilfield chemicals, amine oxides, betaines, polyurethane foam catalysts and epoxy curing agents.

It is also known that certain ADMA products and blends, when used in photopolymerization reactions, can have a beneficial effect on the photopolymerization reaction. For example, see commonly-owned co-pending U.S. patent application Ser. No. 10/511,508, which is hereby incorporated herein in its entirety by reference, which discloses that under certain conditions, ADMA products benefit to photopolymerization reactions.

However, it was heretofore unknown that ADMA products and/or ADMA blends may not be suitable for use in certain applications because they can contain a number of malodorous impurities including trimethylamine (TMA), dimethylamine (DMA), N-methylimine, N,N,N′,N′-tetramethylmethanediamine (bis(dimethylaminomethane)), N-methylformamide, N,N-dimethylformamide, as well as other trace unknown malodorous impurities. These odor-causing impurities cause the ADMA product and/or ADMA blend to have malodorous odors. Furthermore, it was heretofore unknown that ADMA products and ADMA blends that did not have a malodorous odor before initial packaging have been found to develop an unpleasant odor over time when stored in the presence of air. ADMA products and/or ADMA blends that have a malodorous odor have been found to be commercially unusable in some, if not all, applications such as those described above.

Standard purification methods such as, for example, a nitrogen purge at room temperature, treatment with molecular sieves alone, treatment with NaBH₄ alone, or even a combination of these three treatments are not effective, or practical, in a plant-scale process to remove the odor-causing impurities and salts from commercial ADMA products and blends. In addition, a nitrogen purge at 100-130° C. was found to leave some salts and malodorous impurities in ADMA products and blends.

Thus, there exists a need in the art for ADMA products that have reduced levels of malodorous impurities that are useful as, among other things, amine synergists in photopolymerization reactions, and a purification method suitable for the removal of malodorous impurities from impure ADMA products and/or impure ADMA blends. The advantage of such a purification would be the reducing of the offensive smell of impure ADMA products and reducing the amount of any salts present in the impure ADMA product.

### SUMMARY OF THE INVENTION

In some embodiments, the present invention relates to purified ADMA products and the purification of impure ADMA products. The inventors hereof have determined that a water-wash in combination with an inert gas purge at elevated temperatures produces purified ADMA products having reduced salts and malodorous impurities. This simple purification process can be undertaken in a single “pot” or reactor and is easily scaled up for plant use.

Thus, some embodiments of the invention relate to a process for reducing malodorous impurities and salt impurities of an impure alkyldimethylamine (“ADMA”) product comprising:
(a) washing said impure ADMA product with an amount of water to form a water-washed ADMA product; and
(b) purging said water-washed ADMA product with an inert gas while raising and maintaining the temperature of said water-washed ADMA at an elevated temperature and for an amount of time thus forming a purified ADMA product.

The purified ADMA products of the present invention, which are preferably produced by the above process, may contain at most about 20 ppm of DMA, at most about 2 ppm of TMA and at most about 20 ppm of N-methylimine.

In another embodiment, the process may include filtering the impure ADMA before step (a). In another embodiment, the process may include filtering the purified ADMA product after step (b). In another embodiment, the above process may include removing at least a portion of any water present in the water-washed ADMA product by a phase cut. In yet other embodiments, the purification process may include purging with an inert gas while warming the water-washed ADMA after step (a) to about 80° C. with stirring, standing to allow separation of water and ADMA phases, and removing the water phase by a phase cut.

The amount of water added for the water-wash may be in the range of from about 5 to about 20% by weight of the impure ADMA product. In some embodiments, the amount of water is about 10% by weight, on the same basis. The hot purge may be conducted at a temperature in the range of from about 60° C. to about 150° C. In some embodiments, the temperature is in the range of from about 100° C. and about 130° C. The purge gas may be an inert gas such as nitrogen, helium, neon, argon, and xenon. In yet other embodiments, the process may further include adding a masking agent, such as isoamyl acetate, isoamylpropionate, limonene, linolool, β-myrcene, β-phenethyl alcohol and Compounds #80412, #46064 offered commercially by Stanley S. Schoenmann, Inc.

The process of the invention is suitable for use with impure ADMA products that are predominantly individual C₈ to C₁₈ ADMA's, or predominant mixtures thereof, or any combinations thereof. In one embodiment, the ADMA product may be greater than about 95% by weight of C₁₆-alkyldimethylamine. In other embodiments, the ADMA product may be greater than about 95% by weight of C₁₂-alkyldimethylamine. In yet other embodiments, the ADMA product may be predominantly a combination of C₈-ADMA and one other ADMA independently selected from C₁₀-C₂₀ ADMA's.

In one embodiment, the process of purifying impure ADMA products may include (a) washing the ADMA product with an amount of water equal to in the range of from about 10 to 20 wt % of the ADMA to form a water-washed ADMA product; (b) allowing or causing the waster-washed ADMA product to separate into an organic phase and an aqueous phase and recovering the organic phase; and (c) purging the organic phase with an inert gas while heating the organic phase to an elevated temperature in the range of from about 60° C. to about 150° C.; and maintaining the organic phase at the elevated temperature for a specified amount of time thus forming a purified ADMA product. Immediately after the purification process of the invention, the purified ADMA product should contain at most about 20 ppm of DMA, at most about 2 ppm of TMA and at most about 20 ppm of N-methylimine.

In this embodiment, the purification process may further including stirring and purging the impure ADMA while it is being washed by water at a temperature of about 80° C. In some other embodiments, the process may include filtering the ADMA product before step (a) or filtering the ADMA after step (c). The purge gas for the process should be an inert gas, such as nitrogen, helium, neon, argon, and xenon should be use for the purge. If nitrogen is used, the flow rate may be in the range of from about 10 to about 15 standard cubic feet per hour (SCFH). Generally, the purge temperature is in the range of from about 100° C. and about 130° C.

Another aspect of the invention relates to purified ADMA products and/or blends comprising predominantly C₈ to C₁₆ alkyldimethyamines or combinations thereof. The purified ADMA products may contain at most about 20 ppm of DMA, about 2 ppm of TMA and about 20 ppm of N-methylimine. Further, the purified ADMA product and/or blend show no substantial changes in the levels of DMA, TMA and methylamine after stored sealed for no less than about six months under an inert atmosphere. In some embodiment, the purified ADMA products show no substantial changes after storing for no less than about twelve months. In some embodiment, the purified ADMA product contains less than about 0.1 wt % of H₂O. In some other embodiments, the purified ADMA product contains less than about 0.05 wt % of H₂O. In yet other embodiments, the purified ADMA product contains an odor-masking agent such as amyl acetate.

The purified ADMA product of the present invention may comprise predominantly C₈-C₂₀ alkyldimethylamine. In some embodiments, the purified ADMA product contains predominantly C₁₆ alkydimethylamine. In some other embodiments, the purified ADMA product contains predominantly C₁₂ alkydimethylamine. In some other embodiments, the purified ADMA product contains predominantly a combination of C₁₄ and C₁₆ alkyldimethylamines. In some other embodiments, the purified ADMA product contains predominantly a combination of C₈ and other C₁₀ to C₂₀ alkyldimethylamines. In some other embodiments, the purified ADMA product contains predominantly a combination of C₈ and at least one other C₁₀ to C₂₀ alkyldimethylamines.

Another aspect of the invention relates to purified ADMA products made by a process comprising, (a) washing an impure ADMA product having malodorous impurities with an amount of water to form a water-washed ADMA product; and (b) purging the water-washed ADMA product with an inert gas while the temperature of the water-washed ADMA is raised to and maintained at an elevated temperature thus forming a purified ADMA product. Immediately after purification, the purified ADMA product of the present invention comprises at most about 20 ppm of DMA, at most about 2 ppm of TMA, at most about 20 ppm of N-methylimine, and at most 0.1 wt % of residual water. In some embodiments, the amount of washing water is about 10 wt % to about 20 wt % of the impure ADMA product and the elevated temperature is between about 100° C. and about 150° C. In some embodiment, the process may further include filtering the impure ADMA before step (a) to remove solids such as metal halides, ammonium bromides, amine oxides or any combinations thereof. In some embodiments, the process may further include filtering the purified ADMA product after step (b). In some embodiments, the process may include allowing or causing the waster-washed ADMA product to separate into an organic phase and an aqueous phase and recovering the organic phase after step (a) by a phase cut. The aqueous phase typically and preferably comprises the impurities removed from the impure ADMA product, and thus, the phase cut typically also removes soluble impurities from the ADMA water-washed product. These soluble impurities include metal salts, amine oxides, TMA, DMA, N-N-dimethylformamide, N-methylformamide, and any combinations thereof. In some embodiments, the process may include adding a masking agent, such as isoamyl acetate, isoamypropionate, limonene, linolool, β-myrcene, β-phenethyl alcohol and Compounds #80412, #46064 commercially available from Stanley S. Schoenmann, Inc. The purified ADMA products prepared by the present invention may include ADMA products that comprise predominantly an individual C₈ to C₁₆ alkyldimethylamine or any combinations thereof. In some embodiments, the purified ADMA product comprises predominantly C₁₆ alkyldimethylamine. In other embodiments, the purified ADMA product comprises predominantly C₁₂ alkyldimethylamine. In yet other embodiments, the purified ADMA product comprises predominantly a combination of C₈ and at least one other C₁₀ to C₂₀ alkyldimethylamines.

In other embodiments, the present invention relates to the use of purified ADMA products as amine synergists in photopolymerizable reactions. In this embodiment, the present invention is a method of synergizing a photoinitiating reaction comprising combining at least one photopolymerizable monomer and/or oligomer, preferably at least one photopolymerizable monomer, at least one photopolymerization initiator, at least one purified ADMA product, and at least one short chain tertiary amino compound containing at least two electronegative atoms in the molecule to form a photpolymerizable mixture. The photopolymerizable mixture is then contacted with radiation thus producing a photopolymerized article.

Any of the above described aspects and embodiments of the invention can be combined where practical.

### DETAILED DESCRIPTION OF THE INVENTION

The present invention relates to low odor and low salt alkyldimethylamine (ADMA), and blends thereof; particularly, to ADMA products containing reduced amounts of salts and odorous (stinky) impurities; to a process of removing the salts and odor causing impurities in ADMA products; the use of low odor and low salt alkyldimethylamine (ADMA), and blends thereof as amine synergists; and to photopolymerized articles formed from photopolymerization reactions using purified ADMA products as amine synergists.

### Definitions

The term “ADMA” refers to an acronym for alkyldimethylamine. The term “ADMA®” refers to a registered trademark of the Albemarle Corporation. The present invention is applicable to alkyldimethylamine products in addition to those marketed under the Albemarle trademark; unless otherwise specified, ADMA is used herein generically as an acronym for alkyldimethylamine.

The term “ADMA product” or “ADMA blend” can be used interchangeably herein and refer to an alkyldimethylamine containing product, wherein the length of the alkyl chains of the alkyldimethylamines ranges from 8-20 carbon atoms, such chains can be cyclic, straight-chain or branched, and all optionally substituted. It should be noted that “purified ADMA product” and “impure ADMA product” as used herein may be used to refer to either a blend of alkyldimethylamines or a single chain alkyldimethylamine.

“Predominantly” as used when referring to an ADMA product of a single identified carbon chain length means that greater than about 95% by weight of the alkyldimethylamines in that ADMA product is the alkyldimethylamine of the identified carbon chain length. For example, an ADMA product comprising predominantly C₁₆-dimethylamine contains greater than about 95% by weight hexadecyldimethylamine and is sometimes referred to as “ADMA-16”, herein. Correspondingly, ADMA-16 has a distribution of less than about 5% by weight of alkyldimethylamines of other carbon chains, e.g. C₈, C₁₀, C₁₂, C₁₄ and C₁₈, etc.

Additionally, “predominantly” as used when referring to an ADMA blend of two identified carbon chains means that at least about 70% by weight of the alkyldimethyamines in that ADMA blend are the alkyldimethylamines with the two identified carbon chains. For example, an ADMA product comprising predominantly of C₁₄- and C₁₆-dimethylamines contains at least about 70% by weight a combination of tetradecyldimethylamine and hexadecyldimethylamine and is also refer to as “ADMA-1416”.

The term “impurities” with respect to ADMA products and/or ADMA blends pertains to odorless salts and malodorous impurities that can include, but are not limited to, trimethylamine, dimethylamine, N-methylimine (CH₂═NCH₃), N,N,N′,N′-tetramethylmethanediamine (Me₂N—CH₂—NMe₂), N,N-dimethylformamide (HC(O)NMe₂), N-methylformamide (HC(O)NMeH), and other trace malodorous impurities. The purified ADMA product may contain other odorless impurities, for example 1-hexadecene and N-oxide of the alkyldimethylamine; and process impurities such as isopropyl alcohol, methylene chloride, toluene, acetone, etc.

The phrase “impure ADMA product” refers to an ADMA product and/or blend that contain at least one of the malodorous impurities disclosed herein. The impure ADMA product can be further purified according to the present invention to reduce the quantity of odor-causing impurities, thereby resulting in a purified (low odor) product.

The phrase “purified ADMA product” or “low odor ADMA product” refers to an ADMA product and/or blend that has been purified by the process of the present invention, i.e. contains reduced amounts of malodorous impurities compared to the starting impure ADMA product. A purified ADMA product may contain more or less of other non-odorous impurities, and these non-odorous impurities are not generally of concern in regards to the present invention when assessing the purity of a product.

The phrase “low odor” refers to an ADMA product, purified or otherwise, that contains a reduced amount of malodorous impurities, and preferably a reduced amount of salts described herein.

The phrase “phase cut” refers to removal of one phase of a biphasic mixture, in particular, the removal of an aqueous phase from an organic/aqueous phase mixture after phase separation.

The term “inert gas” refers to helium (He), neon (Ne), argon (Ar), krypton (Kr), xenon (Xe), and radon (Rn) and further to substances that do not readily undergo chemical reactions, such as nitrogen (N₂).

The term “inert gas purge” or “nitrogen purge” generally refers to a sub-surface introduction of gas into a liquid ADMA product or a liquid mixture comprising the same. Alternatively the terms refer to blowing gas over the liquid, preferably while the liquid is being stirred.

The term “perfume,” “deodorizer” or “masking agent” refers to odoriferous compounds that possess odors that are generally considered pleasant to the human olfactory. Exemplary masking agents include, but are not limited to, isoamyl acetate, isoamylpropionate, limonene, linolool, β-myrcene, β-phenethyl alcohol and Compounds #80412, #46064 available commercially from Stanley S. Schoenmann, Inc.

### EMBODIMENTS OF THE INVENTION

Without being limited by theory, the inventors of the present invention have discovered that besides the known and unknown malodorous impurities that form during the production of impure ADMA products, one major source of malodorous impurities in impure ADMA products is the reaction of the amines in the ADMA product with oxygen in the air. These impurities accumulate with the passage of time, and result in an ADMA product with a malodorous smell. It has been observed that commercially produced ADMA products can contain a number of malodorous impurities including trimethylamine (TMA), dimethylamine (DMA), N-methylimine (MI), N-methylformamide (MF), N,N,N′,N′-tetramethylmethanediamine (TMMDA), N,N-dimethylformamide (DMF), as well as other trace unknown odorous impurities. Besides the malodorous impurities, commercial ADMA products may contain salts, such as metal salts, ammonium bromide, and N-oxides. These odorless salts do not contribute to the odor of the ADMA product, but can cause degradation of the ADMA product.

The inventors have determined that by washing an impure ADMA product with water followed by purging the water-washed ADMA product with an inert gas, preferably nitrogen, under conditions including elevated temperatures, i.e. above room temperature, at least a portion, preferably substantially all, of the malodorous impurities and at least a portion, preferably substantially all, of the odorless salts present in the impure ADMA product can be effectively removed. The water-washing step of the present invention also preferably removes at least a portion, preferably substantially all, of any low boiling impurities, along with soluble salts and residual solvents that may be present in impure ADMA products. In particular, the water wash step is effective at removing water-soluble, low molecular weight amines and quaternary amines, metal halides, and process impurities such as isopropyl alcohol, methylene chloride, toluene, acetone, dimethylformamide, etc. The inventors hereof have also determined that the subsequent inert gas purge removes at least a portion, preferably substantially all, of any remaining volatile impurities. The inventors hereof have further determined that the inert gas purge of the present invention is effective in removing at least a portion of any residual water, preferably resulting in a purified ADMA product with a water level of less than 0.2 wt %, based on the purified ADMA, from the water-washed ADMA product product. High water content may interfere with downstream applications of the ADMA blends.

It has been determined that reducing the quantity of one or more of the above-described impurities from an impure ADMA product results in a purified ADMA product that does not develop a substantial offensive odor over time if the product is protected from air.

Also, without being limited by theory, the inventors hereof have unexpectedly discovered that the use of these purified ADMA products in combination with certain short chain amines provide a synergistic effect in photopolymerizable reactions, or at least provide improved results as compared to a photopolymerization reaction using the purified ADMA product in the absence of the short chain amine.

### The Purification Process

The purification process of the present invention generally starts with washing an impure ADMA product with a sufficient quantity of water. Typically, deionized water is used, but any other water with similar or higher purity may be used. The amount of water used in the water-washing step of the present invention generally ranges from about 5 to about 20 wt. %, based on the impure ADMA product; and in some embodiments, the amount of water is in the range of from about 7.5 to about 15 wt. %, on the same basis. In other embodiments, in the range of from about 7.5 to about 12.5 wt % water, on the same basis, is used, and in yet other embodiments, about 10 wt. % water, on the same basis, is used in the water-washing step of the present invention. During the water washing step, the mixture comprising the impure ADMA product and the water is preferably stirred at a sufficient speed and for a sufficient duration that the water and impure ADMA product are thoroughly mixed together. Non-limiting examples of speeds and times for a 100 kg sample of impure ADMA product typically includes a stirring speed of below 250 rpm, more typically about 200 rpm, for about an hour.

Typically, the water wash is performed at room temperature. Optionally, the water wash may be performed at an elevated temperature. It has been observed that stirring at an elevated temperature under an inert gas atmosphere, preferably N₂, reduces the formation of a “rag layer” and improves the subsequent phase separation, allowing for a more effective optional phase cut, as described herein. While not wishing to be bound by theory, the inventors hereof believe that a more effective, i.e. cleaner, phase cut has the effect of preventing formation of malodorous or color impurities in the organic phase when the mixture is later exposed to higher temperature. If the water wash is to be performed at an elevated temperature, the temperature of the water-ADMA mixture should be at no higher than about 100° C., typically in the range of from about 60 to about 90° C., and more typically at about 80° C. For added protection from oxidation, the water-ADMA mixture may be continually purged with an inert gas, typically nitrogen, or may have the entrapped air removed by vacuum evacuation.

It is within the scope of the present invention that the impure ADMA product be subjected to the water washing step more that once before being passed to step b) of the process of the present invention. Repeated water washing is especially desirable for impure ADMA products that are particularly malodorous. However, it should be noted that repeated washing might compromise the final yield of the product.

After the water wash has completed, the water and ADMA mixture may be, and is preferably, allowed to separate or caused to separate into an aqueous phase and an organic phase. Phase separation may be achieved by any common methods, e.g., by standing for over a period of time (1 to 2 hours), or by centrifugation. At least a portion, preferably substantially all of the aqueous phase is removed, and the organic phase comprising the water-washed ADMA product is recovered. It has been estimated that the residual water content of the wet or water washed ADMA product is approximately 10,000 ppm.

The next step in the purification process of the present invention is an inert gas purge, preferably N₂, under elevated temperatures. The inert gas purge involves purging, subsurface, an inert gas while the water-washed ADMA product is being stirred and heated to an elevated temperature. It should be noted that the duration of the purge, the flow rate of the purge gas, and the stirring speed all depend on the size of the water-washed ADMA sample through which the inert gas is purged. The purging conditions can be easily determined by those skilled in the art.

It should be noted that it has been observed that ADMA products turn yellow when heated above 120° C. in air. Thus, in a preferred embodiment, the inert gas purging occurs in an inert gas atmosphere or under sub-atmospheric pressures, i.e. under a vacuum, when the conditions under which the inert gas purging occurs include temperatures at or above about 120° C. In another embodiment, the inert gas can be used as a stripping medium. Processes used to strip components from a liquid are well known, and any such process can be used herein such as, for example, counter-current stripping, co-current stripping, etc.

The heating and purging of the water-washed ADMA product may start at the same time, but the temperature of the water-washed ADMA product should be raised slowly to allow entrapped air to escape before the temperature reaches about 80° C. Alternatively the ADMA may be purged for a short duration to remove entrapped air before heating commences. Yet alternatively, the water-washed ADMA may be degassed under a partial vacuum prior to being heated and purged with the inert gas.

Generally, the water-washed ADMA product is heated to from about 60 to about 150° C., more typically to in the range of from about 80 to about 130° C., and even more typically to in the range of from about 100 to about 130° C. To assure uniformity of temperature throughout the water-washed ADMA product, the temperature may be raised in several stages, e.g. to in the range of from about 50 to about 80° C., first, and allowing time for the temperature of the water-washed ADMA product to stabilize and for the low boiling volatiles to be driven off.

Non-limiting examples of inert gasses used as the purging gas in the present invention include nitrogen, helium, neon, argon, and xenon. Typically, either argon or nitrogen is used; more typically, nitrogen is used. The flow rate of the purging gas should be adjusted such that the water-washed ADMA product is thoroughly purged. Non-limiting examples of suitable purge gas flow rates for a 100 g sample of water-washed ADMA product include flow rates in the range of from about 100 to about 200 mL per minute, typically around about 160 mL per minute. Non-limiting examples of suitable purge gas flow rates for a 100 kg sample of water-washed ADMA product include flow rates that are typically in the range of in the range of from about 5 to about 20 standard cubic feet per hour (SCFH) (approximately in the range of from 2.5-10 L per minute), more typically in the range of from about 10 to about 15 SCFH (approximately in the range of from 4.5-7.5 L per minute). Alternatively, the flow rate of the inert purging gas may be changed during the purge period, typically faster at the onset (the first hour) of the purge and slower after afterwards. The duration of the purge may be in the range of from about 2 to about 20 hours. For example, purge times for a 100 g sample are typically less than about 3 hours, and for a 100 kg sample, purge times are typically about 15 hours.

Alternatively, instead of purging with an inert gas, at least a portion of any low boiling volatile impurities and residual water present in the water-washed ADMA product may be removed by vacuum evacuation. When using vacuum evacuation, the other process parameters such as heating temperature, stirring rate, and time may remain substantially the same, and those skilled in the art would know how to adjust these parameters without undue experimentation.

It should be understood that there is no requirement that the purification steps discussed above be followed exactly. For example, the impure ADMA product may be washed more than once, each time with a different amount of water. In another embodiment, the wash water may be removed by hot N₂ purge instead of by phase cut. However, when using this sequence, the inventors hereof have discovered precipitates of ADMA-N-oxides and other salts in the final product. The N-oxides and other salts are generally odorless and will not affect the odor quality of the purified ADMA product, but the since these impurities are solids, they may interfere with the use of the purified ADMA product. Therefore, if the aqueous phase and organic phase are not separated by a phase cut as described herein, it is preferred that the purified ADMA product be filtered to remove at least a portion, preferably substantially all, of any solid impurities contained therein. Thus, it is preferred that a phase cut be used to separate the organic and aqueous phase. It should be noted that there is no need to perform a phase cut after each water-washing step if multiple water-washing steps are used, but in one embodiment, a phase cut is optionally performed after at least one, sometimes all, of the additional water-washing steps.

In one embodiment, the impure ADMA product is filtered prior to the water wash step because the inventors hereof have observed the presence of solid ADMA-N-oxides in stored impure ADMA products that have been exposed to air during storage. These N-oxides are odorless and are readily soluble in water and removable by the water wash step of the present invention. Further, N-oxides of some ADMAs are known detergents, e.g., lauryldimethylamine N-oxide (ADMA-12 N-oxide), which may cause foaming during the water wash step and interfere with the phase separation step. If sufficient amount of solid is present in the starting material, filtering the impure ADMA product before the water wash step is preferred.

After the purification, perfumes or masking agents may be added to impart a favorable scent and extend the “low odor” shelf life of the purified ADMA product. Any masking agents known that are used in ADMA products may be used herein. Exemplary odor masking agents include, but are not limited to, isoamyl acetate, isoamypropionate, limonene, linolool, β-myrcene, β-phenethyl alcohol and Compounds #80412, #46064 commercially available from Stanley S. Schoenmann, Inc. An effective, but not interfering, amount of masking agent may be added to the purified ADMA product. By effective but not interfering amount, it is meant that amount sufficient to mask any malodorous scent present in the purified ADMA product while not affecting the performance of the purified ADMA product. For example, isoamyl acetate, which is also known as pear oil or banana oil, may be added up to about 100 ppm, based on the purified ADMA product.

It is known that oxygen causes the formation of some malodorous impurities in the ADMA products. Thus, to prolong the “low odor” shelf life of the purified ADMA product, the purified ADMA product is preferably kept away from oxygen, excess heat and light during storage. It is recommended that the purified product be stored in airtight containers shielded from light. Any containers that possess such properties may be used, e.g., a sealable metal containers, and opaque bottles. The inventors have found that aluminum alkyl containers are suitable for this purpose. To avoid exposure to air, the purified ADMA product may be vacuum transferred into the storage containers, and, as an added precaution, the purified ADMA products may be stored under an inert gas blanket, i.e. in a container wherein at least a portion, preferably substantially all, of the air present in the container has been displaced and removed by an inert gas.

### Evaluation of the Purification Process

The effectiveness of the purification process of the present invention may be evaluated by measuring the quantity of the malodorous impurities and odorless salts present in the purified ADMA product.

The major odorless salts and malodorous impurities, such as TMA, DMA, MI, TMMDA, DMF and MF, in commercially produced impure ADMA products can easily be determined by well established analytical methods. These analytical methods include, but are not limited to, gas chromatography (GC), headspace GC-MS, liquid chromatography (LC), and inductively coupled plasma emission spectrometry (“ICP”), Karl Fischer water analysis, and proton-NMR. These methods are generally known to those skilled in the art, and discussion may be found in HANDBOOK OF ELEMENTAL SPECIATION: TECHNIQUES AND METHODOLOGY by Cornelis, et al. (2003) John Wiley and Sons, Ltd., INTRODUCTION TO MODERN LIQUID CHROMATOGRAPHY by Snyder and Kirkland, 2nd edition, Wiley-Interscience, and MODERN PRACTICE OF GAS CHROMATOGRAPHY, 2nd edition, Wiley-Interscience. A discussion of Karl Fischer titration technique may be found in “Karl Fischer Volumetric Titration: Theory & Practice Guide”, published by Radiometer Analytical. A copy of the publication may be obtained from the website www.radiometer-analytical.com.

For comparing impure ADMA products to purified ADMA products, not all of the known malodorous impurities need be measured. The inventors have used TMA and DMA as indicators for the effectiveness of the present purification process. These two compounds are known potent malodorous impurities and the methods of detecting and quantifying these compounds are simple, accurate, and known. It should be understood that any other malodorous compound may be selected as an indicator. Alternatively, a survey of a statistically significant number of testers who have evaluated impure ADMA products and purified ADMA products would be equally acceptable for evaluating the success of the method.

### Purified ADMA Products

The present invention also relates to purified ADMA products that comprise predominantly individual C₈ to C₁₈ ADMA products, or any combinations of these ADMA's, i.e. ADMA blends. In some embodiments, the purified ADMA products typically comprise at most about 20 ppm of DMA, at most about 2 ppm of TMA and at most about 20 ppm of N-methylimine, all based on the purified ADMA product. In some embodiments, the purified ADMA product has a residual water content of less than about 1000 ppm, and in other embodiments, the purified ADMA product has a residual water content of less than about 500 ppm, all based on the purified ADMA product. In other embodiments, the purified ADMA product remains low odor with reduced malodorous impurities for a period of in the range of from about 6 to about 12 months. In other embodiments, the purified ADMA product remains low odor with reduced odor impurities for a period of not less than six months.

As stated above, the term “predominantly” when used to refer to a purified ADMA product implies that one alkyldimethylamine having a particular alkyl chain length forms greater than 95 wt % of the product, or two alkyldimethylamines have different alkyl chain lengths form greater than 70 wt % of the product. In one embodiment, the purified ADMA product is composed predominantly of C₁₆ alkyl group; in a second embodiment, of C₁₄ alkyl group; in a third embodiment, of C₁₂ alkyl group; in a fourth embodiment, of C₁₀ alkyl group. In one embodiment, the purified ADMA product is composed predominantly of C₁₈ and C₈ alkyl groups; in another embodiment, predominantly C₁₆ and C₈ alkyl groups; in another embodiment, predominantly C₁₄ and C₈ alkyl groups; in another embodiment, predominantly C₁₂ and C₈ alkyl groups; in another embodiment, predominantly C₁₀ and C₈ alkyl groups; wherein the C₈ alkyl group of the above combinations is not greater than about 25 wt % of the purified ADMA product. In a further embodiment, the purified ADMA product is composed predominantly of a combination of C₁₈ and C₁₆ alkyl groups; in another embodiment, a combination of predominantly C₁₈ and C₁₄ alkyl groups; in yet another embodiment, a combination of predominantly C₁₈ and C₁₂ alkyl groups; in one embodiment, a combination of predominantly C₁₈ and C₁₀ alkyl groups; in a further embodiment, a combination of predominantly C₁₆ and C₁₄ alkyl groups; in another embodiment, a combination of predominantly C₁₆ and C₁₂ alkyl groups; and in yet another embodiment, a combination of predominantly C₁₆ and C₁₀ alkyl groups; in a further embodiment, a combination of predominantly C₁₄ and C₁₂ alkyl groups; in another embodiment, a combination of predominantly C₁₄ and C10 alkyl group; and in yet another embodiment, a combination of predominantly C₁₂ and C₁₀ alkyl groups.

Yet another aspect of the present invention is a purified ADMA product comprising a perfume or odor-masking agent. Exemplary perfumes or odor masking agent that are suitable include, but are not limited to isoamyl acetate, isoamypropionate, limonene, linolool, β-myrcene, β-phenethyl alcohol and Compounds #80412, #46064 commercially available from Stanley S. Schoenmann, Inc. Amounts of masking agents are those described above.

### Products by the Process of the Invention

Still another aspect of the invention is related to an ADMA product which is made by a purification method comprising: (a) stirring an impure ADMA product with in the range of from about 10% to about 20% by weight, based on the impure ADMA product, of H₂O thus forming a water-washed ADMA product; (b) allowing or causing the waster-washed ADMA product to separate into an organic phase and an aqueous phase and recovering the organic phase; and (c) purging the organic phase with an inert gas while heating the organic phase to and maintaining the organic phase at a temperature ranging from about 60-150° C. for an effective amount of time. Inert gasses suitable for use in this embodiment are those described above. In smaller scale purification, i.e. about 100 g, the temperature of the organic phase is generally maintained at in the range of from about 100 to about 120° C. for a duration of time in the range of from about 3 to about 5 hours. In larger scale purification, i.e., about 100 kg, the temperature of the organic phase is generally maintained at in the range of from about 96 to about 130° C. for a duration of time in the range of from about 10 to about 20 hours.

The purified ADMA products of the present invention are suitable for use as amine synergists.

The above description is directed to several means for carrying out the present invention. Those skilled in the art will recognize that other means, which are equally effective, could be devised for carrying out the spirit of this invention. The following examples will illustrate the present invention, but are not meant to be limiting in any manner.

### Purified ADMA Products as Amine Synergists

In this embodiment, the present invention is a method of synergizing a photoinitiating reaction comprising combining i) at least one photopolymerizable monomer and/or oligomer, ii) at least one photopolymerization initiator, iii) at least one purified ADMA product as described herein, and iv) at least one short chain tertiary amino compound containing at least two electronegative atoms in the molecule to form a photopolymerizable mixture. The photopolymerizable mixture is then contacted with radiation thus producing a photopolymerized article. It should be noted that photopolymerized article is used herein in its broadest sense and the photopolymerized article produced depends on the monomer(s) selected and the end use of the article. Thus, photopolymerizable article is meant to encompass coatings, laminates, molded articles, thin film coating for use in paper processing, film coatings, etc.

Typically, the photopolymerizable mixtures are formed by mixing from about 0.5 to about 85 wt %, based on the photopolymerizable mixture, of one, in some embodiments more than one, photopolymerizable monomers such as those described below. Preferably, photopolymerizable mixtures of the present invention are formed by mixing in the range of from about 20 to about 75 wt %, on the same basis, of one, in some embodiments more than one, of such photopolymerizable monomers. Selections within these ranges are typically made for effecting adjustments of viscosity to suit the particular application method to be used. More preferred photopolymerizable mixtures, especially those adapted for use in forming low viscosity web coatings, are formed by using in the range of from about 50 to about 70 wt %, on the same basis, of one, in some embodiments more than one, monomer and/or oligomer, based on the weight of the total composition to be subjected to photopolymerization, i.e. the photopolymerizable mixture.

Photopolymerizable monomers for use in the present invention include any photopolymerizable monomer known. Non-limiting examples of suitable photopolymerizable monomers include acrylates, methacrylates, and the like. Non-limiting examples of such acrylate and methacrylate monomers and oligomers include methyl acrylate, methyl methacrylate, ethyl acrylate, ethyl methacrylate, butyl acrylate, butyl methacrylate, 2-ethylhexyl acrylate, 2-ethylhexyl methacrylate, lauryl acrylate, lauryl methacrylate, cyclohexyl acrylate, cyclohexyl methacrylate, isobornyl acrylate, isobornyl methacrylate, hydroxyethyl acrylate, hydroxyethyl methacrylate, dimethylaminopropyl acrylate, dimethylaminopropyl methacrylate, diethylaminopropyl acrylate, diethylaminopropyl methacrylate, and the like, as well as mixtures of any two or more thereof.

Polyfunctional monomers and oligomers, i.e., compounds or oligomers having more than one alpha-beta-ethylenic site of unsaturation, can also be used in the practice of this invention. Non-limiting examples of such substances include ethylene glycol diacrylate, ethylene glycol dimethacrylate, 1,4-butanediol diacrylate, 1,4-butanediol dimethacrylate, 1,6-hexanediol diacrylate, 1,6-hexanediol dimethacrylate, diethylene glycol diacrylate, diethylene glycol dimethacrylate, dipropylene glycol diacrylate, dipropylene glycol dimethacrylate, tripropylene glycol diacrylate, tripropylene glycol dimethacrylate, trimethylolpropane triacrylate, trimethylolpropane trimethacrylate, glycerol diacrylate, glycerol dimethacrylate, aliphatic urethane diacrylate, aliphatic urethane dimethacrylate, aliphatic urethane triacrylate, aliphatic urethane hexaacrylate, aromatic urethane diacrylate, aromatic urethane dimethacrylate, aromatic urethane triacrylate, polyethylene glycol (400) diacrylate, polyethylene glycol (400) dimethacrylate, polyethylene glycol (600) diacrylate, polyethylene glycol (600) dimethacrylate, ethoxylated neopentylglycol diacrylate, ethoxylated neopentylglycol dimethacrylate, propoxylated neopentyl glycol diacrylate, propoxylated neopentyl glycol dimethacrylate, highly ethoxylated trimethylolpropane triacrylate, highly ethoxylated trimethylolpropane trimethacrylate, ethoxylated bisphenol A diacrylate, ethoxylated bisphenol A dimethacrylate, erythritol tetraacrylate, erythritol tetramethacrylate, amino-modified epoxy diacrylate, epoxy novolac triacrylate, divinylbenzene, 1,3-diisopropenylbenzene, polyester triacrylate, polyester tetraacrylate, polyester hexaacrylate, and diluted acrylic acrylate oligomers such as Ebecryl® 740-40TP, Ebecryl® 745, Ebecryl® 754, Ebecryl® 1701, Ebecryl® 1701-TP20, and Ebecyl® 1710 (all from UCB Chemicals Corporation), and the like, as well as mixtures of any two or more thereof.

If desired, alpha, beta-ethylenically unsaturated carboxylic acids can be used in conjunction with acrylate and/or methacrylate monomers, typically for the purpose of providing improved adhesion to certain substrates. Examples of such acids include methacrylic acid, acrylic acid, itaconic acid, maleic acid, beta-carboxyethyl acrylate, beta-carboxyethyl methacrylate, and the like, as well as mixtures of any two or more thereof. Preferred photopolymeriable monomers of this invention are, however, devoid of such carboxylic acids except as may be present as impurities or as residuals from manufacture.

Preferred photopolymerizable monomers for use in the practice of this invention include tripropylene glycol diacrylate, trimethylol propane tetraacrylate, ethoxylated trimethylol propane tetraacrylate, propoxylated neopentyl glycol diacrylate, hexanediol diacrylate, and the like, as well as mixtures of any two or more thereof.

The photopolymerizable mixture formed in the present invention comprises at least one photoinitiator, or mixtures of photoinitiators. The photoinitiator is typically added in an amount of in the range of from about 0.01 to about 20 parts by weight, preferably in the range of from about 0.05 to about 15 parts by weight, per 100 parts by weight of the photopolymerizable monomer(s). More preferably, the photoinitiator is added in an amount in the range of from about 0.01 to 10 parts by weight, most preferably in the range of from 0.05 to 5 parts by weight, per 100 parts by weight of the photopolymerizable monomer(s).

The purified ADMA products used in this embodiment of the present invention include any of those described herein, which have been purified by the purification process disclosed herein. The purified ADMA product is typically added in an amount ranging from about 0.1 parts to about 15 parts, based on the weight of the photopolymerizable mixture. In preferred embodiments, the photopolymerizable mixture is formed by mixing in the range of from about 0.5 parts to about 10 parts, on the same basis, of a purified ADMA product with the other components i), ii), and iv), more preferably in the range of from about 1 part to about 5 parts, on the same basis.

The present invention can be practiced with various photopolymerization initiators, simply referred to herein as initiators sometimes. These initiators are typically added in an amount of in the range of from 0.01 to 10 parts by weight, preferably in the range of from 0.05 to 5 parts by weight, per 100 parts by weight of the photpolymerizable monomer(s).

Photopolymerizable initiators suitable for use herein include hydrogen Type I (unimolecular fragmentation type) initiators, such as alpha-diketone compounds or monoketal derivatives thereof (e.g., diacetyl, benzil, benzyl, or dimethylketal derivatives); acyloins (e.g., benzoin, pivaloin, etc.); acyloin ethers (e.g., benzoin methyl ether, benzoin ethyl ether, benzoin propyl ether, etc.), acyl phosphine oxides, and other similar Type I initiators, including mixtures of any two or more such initiators. Similarly, Type II (abstraction-type) initiators can be used. Non-limiting examples of suitable Type II initiators include xanthone, thioxanthone, 2-chloroxanthone, benzil, benzophenone, 4,4′-bis(N,N′-dimethylamino)benzophenone, polynuclear quinones (e.g., 9,10-anthraquinone, 9,10-phenanthrenequinone, 2-ethyl anthraquinone, and 1,4-naphthoquinone), or the like, as well as mixtures of any two or more thereof. Preferred Type I initiators include ketals such as benzyl dimethyl ketal. Preferred Type II initiators include hydrogen quinones such as benzoquinone and 2-ethyl anthraquinone. Mixtures of Type I and Type II initiators can also be used.

The inventors hereof have unexpectedly discovered that the use of the purified ADMA products in combination with certain short chain amines provide a synergistic effect in the present method, or at least provide improved results as compared to photopolymerization reactions using the purified ADMA product in the absence of the short chain amine. For example, the combination of a short chain amine in the form of, e.g., N-[3-(dimethylamino)propyl]-N,N′,N′-trimethyl-1,3-propanediamine (Polycat 77; Air Products, Inc.), or 2,2′-oxybis[N,N-dimethylethanamine] (DABCO BL-19; Air Products, Inc.), methyldiethanolamine, hydroxyethyl morpholine, or preferably N,N-dimethyl-4-morpholineethanamine (DABCO XDM; Air Products, Inc.), when used in combination with the above purified ADMA products and 2-hydroxy-2-methyl-1-phenylpropane-1-one, provide synergistic results. N,N-dimethyl-4-morpholineethanamine, when used in combination with dodecyldimethylamine and 2-hydroxy-2-methyl-1-phenylpropane-1-one, has been shown to be effective at a lower percentage as compared to methyldiethanolamine.

In the practice of the present invention, in the range of from about 0.1 parts to about 15 parts, based on the weight of the photopolymerizable mixture, of at least one, preferably only one, short chain tertiary amino compound (referred to as a short chain amine herein also) is mixed with the other ingredients to form the photopolymerizable mixture. In preferred embodiments, in the range of from about 0.5 parts to about 10 parts, on the same basis, of the short chain amine is used, and in a more preferred embodiment, in the range of from about 1 part to about 5 parts, on the same basis.

Short chain amines suitable for use in the present invention are tertiary amino compounds containing at least two electronegative atoms in the molecule, at least one of which is a tertiary nitrogen atom and another of which is an oxygen atom or a tertiary nitrogen atom, and wherein the electronegative atoms are bonded only to short chain alkyl or alkylene groups (e.g., C₁-C₃ alkyl or alkylene groups), and wherein the compound has a total of at least 4 and preferably at least 6 abstractable hydrogen atoms in positions alpha to at least some of the electronegative atoms in the compound. To illustrate, N-[3-(dimethylamino)propyl]-N,N′,N′-trimethyl-1,3-propanediamine has three electronegative atoms and a total of 9 abstractable hydrogen atoms in the molecule. 2,2′-Oxybis[N,N-dimethylethanamine] has three electronegative atoms and a total of 8 abstractable hydrogen atoms in the molecule. N,N-dimethyl-2-morpholinoethanamine has two electronegative atoms and a total of 8 abstractable hydrogen atoms in the molecule. N-hydroxyethylmorpholine has two electronegative atoms and a total of 6 abstractable hydrogen atoms in the molecule. A short chain amine having the requisite number of abstractable hydrogen atoms will cause polymerization to occur when used with benzophenone in a mixture with epoxyacrylate diluted with tripropylene glycol diacrylate in a 35:65 wt ratio on exposure of the mixture UV light at 254 nonometers. The forgoing illustrative short chain amines make clear that the short chain alkylene groups can be part of a non-cyclic compound or of a cyclic compound. Thus for example in N-[3-(dimethylamino)propyl]-N,N′,N′-trimethyl-1,3-propanediamine, the alkylene group (the propane moiety) is in a non-cyclic compound. In contrast, in N-hydroxyethylmorpholine there are two alkylene (ethylene) groups in the morpholine moiety, which groups form a cyclic morpholine ring with an oxygen atom and a nitrogen atom, as well as an open chain alkylene group (the ethyl moiety in the N-hydroxyethyl group).

Among the various types of suitable short chain tertiary amino compounds suitable for use herein are compounds represented by the formula:
R—(CH₂)ₙ—NR¹R²
where
A) R is (i) a dialkylamino group in which each alkyl is, independently, a C₁-C₃ primary alkyl group; (ii) an N-alkylpiperazinyl group in which the alkyl is a C₁₋₃ primary alkyl group, or (iii) a morpholino group; (iv) a C₁-C₃ alkylhydoxy group.
   R¹ is an alkylhydoxy group or a dialkylamino group in which each alkyl is, independently, a C₁₋₃ primary alkyl group;
   R² is (i) a dialkylamino group in which each alkyl is, independently, a C₁-C₃ primary alkyl group; (ii) an alkyleneamino group in which alkylene is a C₁-C₃ alkylene group and the amino is a dialkylamino group in which each alkyl is, independently, a C₁-C₃ primary alkyl group; (iii) an alkyleneaminoalkyleneamino group (—R—N(R)—R—NR₂) in which each alkylene is, independently, a C₁-C₃ alkylene group, the amino between the alkylenes is a C₁-C₃ primary alkylamino group, and the other amino is a dialkylamino group in which each alkyl is, independently, a C₁-C₃ primary alkyl group; (iv) an alkyleneoxyalkyleneamino group (—R—O—R—NR₂) in which each alkylene is, independently, a C₁-C₃ alkylene group, and the amino is a dialkylamino group in which each alkyl is, independently, a C₁-C₃ primary alkyl group; or (v) an alkyleneoxyalkyleneoxyalkyleneamino group (—R—O—R—O—R—NR₂) in which each alkylene is, independently, a C₁-C₃ alkylene group, and the amino is a dialkylamino group in which each alkyl is, independently, a C₁-C₃ primary alkyl group;
      or where
B) R is (i) a dialkylamino group in which each alkyl is, independently, a C₁-C₃ primary alkyl group; (ii) an N-alkylpiperazinyl group in which the alkyl is a C₁-C₃ primary alkyl group, or (iii) a morpholino group; and R¹ and R² taken together is (i) an N-alkylpiperazinyl group in which the alkyl is a C₁-C₃ primary alkyl group, or (ii) a morpholino group.

In addition to the above, many other types of short chain amines can be used in the present invention. In general, the short chain amine will typically consist of one or more tertiary amino groups, one or more ether oxygen atoms, and/or one or two hydroxyl groups linked to each other by C₁-C₃ aklylene groups, such that there are at least two tertiary amino groups or at least one tertiary amino group and at least one ether oxygen atom or at least one hydroxyl group linked together in this fashion, and such that the compound has a total of at least 4 and preferably at least 6 abstractable hydrogen atoms in positions alpha to at least some of the electronegative atoms in the compound. The tertiary amino group(s) when not part of a cycloaliphatic ring system are di(C₁₋₃ alkyl)amino or mono(C₁₋₃ alkyl)amino group(s) depending on whether the tertiary amino group is a terminal group or an internal group.

A few non-limiting examples of suitable short chain amines include N,N,N′-trimethyl-1,2-ethanediamine, N,N,N′,N′-tetramethyl-1,2-ethanediamine, N,N,N′-trimethyl-1,3-propanediamine, N,N,N′,N′-tetramethyl-1,3-propanediamine, N-[2-(dimethylamino)ethyl]-N,N′,N′-trimethyl-1,2-ethanediamine, N-[3-(dimethylamino)propyl]-N,N′,N′-trimethyl-1,3-propanediamine, 1,4-dimethylpiperazine, 2,2′-oxybis[N,N-dimethylethanamine], 3,3′-oxybis[N,N-dimethylpropanamine], 4-[2-(dimethylamino)ethyl]morpholine (a.k.a. N,N-dimethyl-2-morpholinoethanamine), 4-[3-(dimethylamino)propyl]morpholine, and the homologs of the foregoing amines in which some or all of the methyl groups are replaced by ethyl or propylgroups, triethylenediamine, 4,4′-(oxydi-2,1-ethanediyl)bismorpholine, N-hydroxyethylmorpholine, and N-hydroxypropylmorpholine.

In some embodiments, other components, or additives, can also be used to form the photopolymerizable mixture. For instance, pigments and dyes can be used, and in some embodiments are preferably used, in forming the photopolymerizable mixture. Non-limiting examples of pigments and typical amounts used include phthalocyanine blue (in the range of from 1 to 40 wt %), titanium dioxide (in the range of from 10 to 30 wt %), carbon black (in the range of from 1 to 60%) or other organic or inorganic pigments employed in the art. Optionally, dyes such as nigrosine black or methylene blue may be used to enhance color or tone (in the range of from 1 to 15 wt %). All weight percents are based on the weight of the photopolymerizable mixture.

Light stabilizers are another type of additive that can be, and in some embodiments are preferably, used in forming the photopolymerizable mixture. Non-limiting examples of such light stabilizers include 2-hydroxybenzophenones such as 2,2′-dihydroxy-4,4′-dimethoxylbenzophenone, 2-(2-hydroxyphenyl)benzotriazoles such as 2-(2′-hydroxyphenyl)benzotriazole, sterically-hindered amines such as bis(2,2,6,6-tetramethyl-4-piperidyl)sebacate or bis(2,2,6,6-tetramethyl-4-piperidyl)succinate, oxamides such as 4,4′-dioctyloxyanilide, acrylates such as ethyl α-cyano-β,β-diphenylacrylate or methyl α-carbomethoxycinnanamate, and nickel complexes such as the nickel complex of 2,2′-thiobis[(1,1,3,3-tetramethylbutyl)phenol. Typically the amount of light stabilizer used will be in the range of from about 0.02 to about 5 wt %, based on the weight of the photopolymerizable mixture, depending upon the particular type of light stabilizer employed.

Still another type of additive that can be used, and in preferred embodiments is used, in forming the photopolymerizable mixture is one or more radical scavengers. Non-limiting examples of suitable radical scavengers for such use include hydroquinone, hydroquinone methyl ether, p-tert-butylcatechol, quinoid compounds such as benzoquinone and alkyl-substituted benzoquinones, as well as other radical scavenger compounds known in the art. Typically these components will be used in amounts in the range of from about 100 ppm to about 2 percent by weight of the photopolymerizable mixture.

Adhesion promoters constitute yet another type of additive component that can be used in the present invention, and in some preferred embodiments is used. Adhesion promoters suitable for use herein are typically silane derivatives such as gamma-aminopropyltriethoxysilane (DOW A-1100) and equivalent substituted silane products; acid functionally-substituted resins; oligomers or monomers, such as partial esters of phosphoric acid, maleic anhydride, or phthalic anhydride, with or without acrylic or methacrylic unsaturation; and dimers and trimers of acrylic/methacrylic acid. If adhesion promoters are used, the preferred types are other than alpha,beta-ethylenically unsaturated carboxylic acids. If and when used, the concentration of adhesion promoter is determined empirically by adhesion tests. In general, however, amounts are often in the range of from about 0.1 to about 20 wt %, and in more preferred cases in the range of from about 2 to about 10 wt %, based on the weight of the photopolymerizable mixture.

The photopolymerizable mixture formed by mixing the above components, optional and otherwise, is then contacted with radiation. The contacting of the photopolymerizable mixture with radiation effects the synergistic photopolymerization of the photopolymerizable monomer(s) thus forming a photopolymerized article. There are various ways of effecting the photopolymerization of the photopolymerizable monomer(s). For example, the photopolymerizable mixture can be contacted with the radiation while it is a thin coating on a traveling web. Alternatively, the photopolymerizable mixture can be contacted with the radiation when it is a coating or laminate on a substrate. Another variant is where the photopolymerizable mixture is contacted with the radiation when it is an article or shape in a mold. In these and other modes of operation, the exposure to radiation for effecting photopolymerization can be continuous or intermittent.

In one embodiment, the photopolymerization of the monomers and/or oligomers, such as those suitable for films having a thickness of about 2 mils or less that are formed by coating systems operating at high linear speeds, are exposed, i.e. contacted, with the radiation for only an extremely short period of time. For example those films that are used in the manufacture of thinly-coated papers or thin high grade card or paperboard stock used in producing magazine covers, brochures, corporate annual reports, folders, and the like typically employ high-speed coating systems and are typically applied to paper webs traveling at speeds of about 10 feet per second. Therefore, the photopolymerizable mixture is contacted with radiation for as little as in the range of from about 0.005 to about 0.02 seconds. Thus, the amine coinitiators, i.e. synergists, used must function extremely rapidly while at the same time becoming fixed within the polymerized coating without discoloration and without undergoing or causing other types of degradation within the thin film.

An advantageous feature of such concurrent production and in situ application or bonding of such thin photopolymerized coatings on a traveling paper or thin paperboard or card stock is that no other operations such as washing or drying are required. Indeed, it is preferable to conduct the concurrent production and in situ application or bonding of not only such thin photopolymerized coatings on a traveling paper or thin paperboard or card stock, but also the production of other articles, such as coatings or laminates, without use of washing or drying steps. Thus, in one embodiment, the present method can include a step of washing the photopolymerized article, and in another embodiment, these steps are unnecessary. Printed matter, decorations, or the like may thereafter be applied to the photopolymerized article, coating, or laminate using conventional techniques, if desired.

The photopolymerized compositions of this invention can themselves constitute photopolymerizable inks or coatings applied as printed, decorative, or pictorial matter on a substrate and then photopolymerized in place. In this embodiment of the invention the photopolymerizable mixture will include one or more pigments, dyes, or other color-producing substances so that permanent printed matter is formed upon exposure to radiation to effect photopolymerization.

In effecting photopolymerization pursuant to this invention either coherent or non-coherent radiation can be employed. Various sources of such radiation can be employed, such as an ion gas laser (e.g., an argon ion laser, a krypton laser, a helium:cadmium laser, or the like), a solid state laser (e.g., a frequency-doubled Nd:YAG laser), a semiconductor diode laser, an arc lamp (e.g., a medium pressure mercury lamp, a Xenon lamp, or a carbon arc lamp), and like radiation sources. Exposure sources capable of providing ultraviolet and visible wavelength radiation (with wavelengths typically falling in the range of from 300-700 nm) can also be used for the practice of the present invention. Preferred wavelengths are those that correspond to the spectral sensitivity of the initiator being employed. Preferred radiation sources are gas discharge lamps using vapors of mercury, argon, gallium, or iron salts and utilizing magnetic, microwave or electronic ballast; such lamps commonly are medium pressure mercury lamps, or lamps made by Fusion Systems (i.e., D, H, and V lamps).

Exposure times can vary depending upon the radiation source, and photoinitiator(s) being used. For preferred high-speed applications such as in forming thin coatings on paper webs traveling at high linear speeds, times in the range of from about 0.005 to about 0.015 second are preferred. In photopolymerization operations in which the photopolymerizable mixture being polymerized is either stationary or moving slowly as on a conveyor belt, longer exposure times (e.g., in the range of from about 0.2 to about 0.4 seconds) can be used.

Various photopolymerized articles, which can be molded into various shapes either before or after exposure to the radiation, can be produced by use of this embodiment of the present invention. For example, the photopolymerized article can be printed matter on a substrate such as paper, cardboard, or plastic film, etc.; manufactured articles such as handles, knobs, inkstand bases, small trays, rulers, etc.; and coatings or laminates on substrates such as plywood, metal sheeting, polymer composite sheeting, etc. As noted above, thin-coated paper and coated card or thin paperboard stock where the coatings are up to about 2 mils in thickness constitute preferred articles produced pursuant to this invention. In additional preferred embodiments, the synergized photopolymerization method is used in the preparation of thin paper coatings (e.g., 3 to 10 microns) over print or film, applied by gravure, flexo, rod, or offset press; involves applying the photopolymerizable mixture as coatings and/or inks (e.g., in the range of from 15 to 35 microns) by roller coater or curtain coater over flooring (e.g., vinyl sheet goods) or wood panels; and involves applying the photopolymerizable mixture as coatings and/or inks (e.g., in the range of from 10 to 20 microns) applied by flat bed or rotary screen print for labels and packages.

The above description is directed to several embodiments of the present invention. Those skilled in the art will recognize that other means, which are equally effective, could be devised for carrying out the spirit of this invention. It should also be noted that preferred embodiments of the present invention contemplate that all ranges discussed herein include ranges from any lower amount to any higher amount. For example, the amount of adhesion promotes can include amounts are often in the range of from about 0.1 to about 2 wt %, in the range of from about 0.1 to about 10 wt %, in the range of from about 10 wt. % to about 20 wt. %, etc. The following examples will illustrate several embodiments of the present invention, but are not meant to be limiting in any manner.

### EXAMPLES

The above described process of the present invention is applicable for the purification of impure ADMA products; particularly C₈ to C₁₈ ADMA products, either as an individual ADMA or as a blend of ADMA's. Table 1 below summarized the analyses of residual impurities found in ADMA samples before and after being purified by the process of the present invention. The “controls” were the impure ADMA products. The examples demonstrate that water washing (Example 1) alone or nitrogen purge at elevated temperature (130° C.) (Example 2) alone of impure ADMA-16 products only removed approximately half of the impurities as evidenced by the reduction of DMA from 87 ppm in control sample to about 42 ppm in the water washed only sample, and to about 45 ppm in the nitrogen purged only sample. Similarly, the process which used NaBH₄, and a mild reducing agent, in combination with a molecular sieve, nitrogen purge, and purified ADMA product filtration at room temperature (Example 3) removed only half of the low boiling volatiles, as evidenced by the reduction of DMA from 87 ppm in the control sample to 54 ppm, and about 75% of the metal halide salts. The processes of the present invention, either in the lab scale or in the pilot plant scale depending on the particular Example, removed higher percentages of the impurities. For example, on a laboratory scale process (H₂O/N₂ purged at 100° C. for 3 hrs, Example 4) to purify impure ADMA-16, approximately 80% of the DMA and substantially all of the TMA were removed. The process was effective in reducing the content of the other organic impurities such as MI, TMMDA, DFM, and MF, as demonstrated in the Examples, because only approximately in the range of from 20-30% of these organic impurities remained in the purified ADMA product. Further, the lab scale process was also effective in reducing the concentration of salts, as evidenced by the reduction of Br⁻ ion from 5.1 ppm to <0.1 ppm and a 10-fold reduction of Na⁺ and Fe⁺⁺ ions. The pilot plant scale process (H₂O/N₂ purged at 120° C. for 15 hrs, Example 5) was even more effective because, as evidenced by the results of the Examples, substantially all of the malodorous impurities were removed from impure ADMA-16. While not wishing to be bound, the inventors hereof hypothesize that the greater effectiveness of the pilot plant procedure is the result of a longer purge time used therein.

The purification process worked equally well for impure ADMA-12. In the lab scale purification experiment (H₂O/N₂ purged at in the range of from 100-110° C. for 3hrs, Example 6), the content of TMA, DMA and N-methylimine was significantly reduced.

Additionally, the purified ADMA products produced in the Examples herein, both ADMA-16 and ADMA-12, were colorless, and contained below 100 ppm of water. Further, the yield of purified ADMA products was greater than about 95%.

TABLE 1
PURIFICATION OF ADMA'S

| | Fe⁺⁺ | Na⁺ | Br⁻ | TMA | DMA | MI | TMDA | DMF | MF | H₂0 | |
|---|---|---|---|---|---|---|---|---|---|---|---|
| | ppm | ppm | ppm | ppm | ppm | ppm | ppm | ppm | ppm | ppm | Yield % |
| ADMA-16 | | | | | | | | | | | |
| Control* | 0.11 | 1.6 | 5.1 | 4 | 87 | 25-27 | 450-500 | 250 | 100 | ND | NA |
| H₂0 wash | ND | ND | ND | 0 | 42 | ND | ND | ND | ND | ND | ND |
| Example 1 | | | | | | | | | | | |
| N₂ 125° C. 3 hrs | ND | ND | ND | 1 | 45 | ND | ND | ND | ND | ND | ND |
| Example 2 | | | | | | | | | | | |
| NaBH₄/MS/N₂ | 0.012 | 0.43 | 0.17 | <1 | 54 | ND | ND | ND | ND | 300 | ND |
| 22° C. | | | | | | | | | | | |
| Example 3 | | | | | | | | | | | |
| H₂0/N₂ 100° C. | 0.093 | 0.11 | <0.1 | 0 | 17 | 17 | 100 | 70 | 20 | 35 | 96 |
| 3 hrs Example 4 | | | | | | | | | | | |
| H₂0/N₂ 120° C. | ND | ND | ND | 0 | 2 | 5 | ND | ND | ND | 97 | 98 |
| 15 hrs Example 5 | | | | | | | | | | | |
| ADMA-12 | | | | | | | | | | | |
| Control | ND | ND | ND | ˜5 | 29 | 8-9 | ND | ND | ND | ND | NA |
| H₂0/N₂ 100-110° C. | ND | ND | ND | 0 | 11 ± 3 | 3-5 | ND | ND | ND | 27 | >95 |
| 3 hrs Example 6 | | | | | | | | | | | |

*as prepared by Albemarle at Magnolia, Arkansas
ND = not determined
NA = not applicable

The stability of purified ADMA products may be similarly evaluated by determining the content of malodorous impurities in stored samples overtime. Samples of a purified ADMA-16 product (Example 5) and purified ADMA-12 (Example 6) were allowed to age in either nitrogen or in air and in either glass bottle or metal containers, as indicated in the Examples. After the set time period, the samples were observed and analyzed for TMA, DMA and MI. The results are reported in Table 2 below. There were no significant changes in the samples that were aged in nitrogen, and these samples had not developed any off-odor and the content of malodorous impurities remained low. There were observable changes to the samples that were stored in air and exposed to light, and these samples developed malodorous smells. The level of malodorous impurities increased, for example the amount of DMA in the ADMA-16 product increased from 2 ppm as purified to 91 ppm four months later. Additionally, a precipitate developed in the aged ADMA-16 sample. The precipitate was evaluated and determined to be ADMA-N-oxide. Also, 700 ppm, based on the precipitate and determined by GC-MS, of 1-hexadecene was observed in a 5-month air-aged sample of ADMA-16. The result of the aging study is reported in Table 2 below.

TABLE 2
AGING OF PURIFIED ADMA

| | TMA | DMA | MI | |
|---|---|---|---|---|
| | ppm | ppm | ppm | Misc |
| ADMA 16 | | | | |
| As purified (pilot | 0 | 2 | 5-10 | |
| plant) | | | | |
| 6 months under N₂ | 0.03 | 5 | ˜5 | |
| 4 months in air | 1.4 | 91 | 25 | |
| 5 months in air | | | | 700 ppm 1-hexdecene |
| ADMA 12 | | | | |
| As purified | <1 | 11 ± 3 | 3-5 | |
| 5 months under N₂ | <1 | 8 | ND | |
| 4 months under N₂ | <1 | 8.2 | 3 | |

ND = not determined

### Example 1

### Water Wash of ADMA-16

About 20 g of impure ADMA-16 was weighed and charged to a pot at room temperature. About 10 wt. %, based on the impure ADMA-16, was added to the impure ADMA-16 and the mixture stirred for 5 minutes. The mixture was allowed to sit for 30 minutes while the phases separated. The bottom aqueous layer was removed by a phase cut, and the organic phase, the upper layer, was recovered. The organic phase, comprising water-washed ADMA-16, was analyzed for DMA by GC and shown to contain 42 ppm of DMA. The result is reported in Table 1, above.

### Example 2

### Nitrogen Purge of ADMA-16

About 20 g of impure ADMA-16 was weighed and charged to a pot at room temperature. N₂(g) at a flow rate of about 160 mL/min was passed subsurface into the ADMA-16 for about 10-15 minutes. The ADMA-16 was then heated to and maintained at about 125° C. with continuous nitrogen purge for 3 hours. The N₂-purged sample remained colorless and clear. The nitrogen-purged sample was analyzed for DMA by GC and was shown to contain 45 ppm of DMA. The result is reported in Table 1, above.

### Example 3

### NaBH4/Molecular Seive/N₂ Purge at Room Temperature of ADMA-16

Into a flask equipped with a stirrer and an N₂ (g) purge, 0.75 g NaBH₄ and 1.5 kg activated #4 molecular sieves (MS) were added. The flask was charged with 15.5 kg impure ADMA-16 with stirring and an N₂ (g) purge at 22° C. After purging and stirring for 24 hours, the solution was filtered, yielding a purified ADMA-16. The purified sample was analyzed. The purified ADMA-16 contained 0.012 ppm of Fe⁺⁺, 0.43 ppm of Na⁺, 0.17 ppm of Br⁻, <1 ppm of TMA, and 54 ppm of DMA. The purified sample also contained less than 300 ppm of residual H₂O. The result is reported in Table 1, above.

### Example 4

### Purification of ADMA-16 in a Laboratory

22.2 g of impure ADMA-16 was charged to a pot at 22° C. 2.22 g H₂O was added to the pot and the mixture was stirred for 5 minutes. The mixture was allowed to sit for 30 minutes while the phases separated. The bottom aqueous layer, about 2.09 g, was removed, and the organic phase, the upper layer, was recovered. N₂(g) at a flow rate of about 160 mL/min was passed subsurface into the organic phase, comprising water-washed ADMA-16, for about 10-15 minutes. The organic phase was then heated to and maintained at 100-130° C. with continuous nitrogen purge for 3 hours. The purified ADMA-16 was colorless and the recovery was around 96%, based on the impure ADMA-16. Both the impure ADMA-16 and the purified ADMA-16 were analyzed for content of salts, and malodorous impurities by GC, LC, ICP and Karl Fischer analyses.

The impure ADMA-16 contained about: 0.11 ppm of Fe⁺⁺, 1.6 ppm of Na⁺, 5.1 ppm of Br⁻, 4 ppm of TMA, 87 ppm of DMA, 25-27 ppm of MI, 450-500 ppm of TMDA, 250 ppm of DMF, 100 ppm of MF. The purified ADMA-16 contained about: 0.093 ppm of Fe⁺⁺, 0.11 ppm of Na⁺ and <0.1 ppm of Br⁻, 35 ppm of and H₂O, 0 ppm of TMA, 17 ppm of DMA, 17 ppm of MI, 100 ppm of TMDA, 70 ppm of DMF, 20 ppm of MF and 35 ppm of H₂O.

The result is reported in Table 1, above.

### Example 5

### Purification of ADMA-16 in a Pilot Plant

A 100 L reactor equipped with a stirrer was purged with nitrogen. To the reactor 66.4 kg ADMA-16 was added, and deionized water (10% by weight of ADMA-16) was added. The mixture was stirred for 1 hr at 80° C. at 250 rpm. The mixture was then allowed to sit for 1-2 hours, to allow the organic and aqueous phases to separate. The bottom aqueous phase was removed (6402 g aqueous phase and 227 g rag layer), and the organic phase, the upper layer, was recovered. Stirring at 200 rpm was restarted and the organic phase comprising the water-washed ADMA-16 was heated to and maintained at about 120° C. with a subsurface N₂(g) purge for 1 hr at a flow rate of 15 SCFH (standard cubic feet per hour). After the temperature reached 120° C. the temperature of the organic phase was maintained at 120° C. and stirred at 200 rpm for 15 hrs with subsurface feed of N₂(g) at 10 SCFH. After 15 hours, the organic phase was cooled to 25° C. and vacuum transferred to a clean, dry 26 gallon aluminum alkyl container. 65.6 kg (98% yield based on the impure ADMA-16) of purified ADMA-16 was recovered. The purified sample contained, as determined by GC, 2 ppm of DMA, 5 ppm of MI and undetected amounts of TMA, and contained as determined by Karl Fischer analyses less than 100 ppm of H₂O.

The result is reported in Table 1, above.

### Example 6

### Purification of ADMA-12 in a Laboratory

A reactor equipped with a stirrer was purged with nitrogen. To the reactor 20.09 g ADMA 12 was transferred and deionized water (10% by weight of ADMA-12) was added. The mixture was stirred for 15 min at 22° C. and then allowed to sit for 2 hours, for the phases to separate. The bottom aqueous phase (1.96 g) was removed, and the organic phase, the upper layer, was recovered. Another 2.0 g water was added to the reaction vessel and the mixture stirred for 10 minutes. The mixture was allowed to sit for 30 minutes for the phases to separate and the bottom aqueous phase (2.01 g) was removed, and the organic phase, the upper layer, was recovered. The resulting organic phase comprising the water-washed ADMA-12 (about 18.0 g) was charged into a reactor; while the ADMA-12 was still at 22° C., and the air was removed by a slow N₂(g) purge (˜160 mL/minute) for 10 minutes. The organic phase was slowly warmed to about 80-90° C. and then to 100-110° C. with a continuous N₂(g) purge (˜160 mL/minute) over a period of 3 hours. The organic phase was then cooled to 22° C. and purified ADMA-12 was recovered in >95% yield, based on the impure ADMA-12. The impure ADMA-12 and the purified ADMA-12 were analyzed for salts, malodorous impurities and H₂O content. It was found that the impure ADMA-12 contained about: 5 ppm of TMA, 29 ppm of DMA and 8-9 ppm of MI. The purified ADMA-12 contained: non-detectable amounts of TMA, about 11±3 ppm of DMA, about 3-5 ppm of MI and the water content was 27 ppm.

The result is reported in Table 1, above.

### Example 7

### Effect of Heating ADMA in Air

Into each of several flasks equipped with a stirrer was charged a 15.0 g or 11.5 g sample of commercial ADMA-16. Sample 1 (15.0 g) was heated to 100° C. under a vacuum for 1 hour under constant stirring, and no color change was observed. Sample 2 (11.5 g) was heated to 135° C. under a vacuum for 1 hour under constant stirring, and no color change observed. Sample 3 (15.0 g) was heated at 120-125° C. under constant stirring in air for 1 hour, and the sample turned slightly yellow. Sample 4 (15.0 g) was heated in air at 130-150° C. under constant stirring for 2 hours, and the sample turned yellow.

### Example 8

### Stability of Purified ADMA-16 Under Nitrogen and in Air

Fractions of the purified ADMA-16 prepared in Example 5 were used for this Example.

65.6 kg of the purified sample was placed in a metal alkyl cylinder and stored under nitrogen. Approximately 50 ml of the purified ADMA-16 was placed into 4 oz glass bottles and stored in air at a temperature of about 22° C.

A 2 ml sample of the fraction stored under air was taken at about four months after purification to analyze by GC for impurities. A second 2 ml sample of the fraction stored under air was taken at round five months after purification to specifically analyze by GC-MS to identify and quantify any olefins present therein and proton NMR to identify and quantify a white precipitate that was formed during storage.

A 50 ml sample of the fraction that had been stored in the original metal container was taken at about six months after purification and analyzed by GC for the content of the impurities.

The sample that had been stored for four months in a glass bottle under air developed a strong amine odor and was found to contain 1.4 ppm of TMA, 91 ppm of DMA and 25 ppm of MI. The white precipitate was identified as ADMA-16-N-oxide. In the sample that was tested after 5 months in the glass bottle, 1-hexadecene (a C₁₆-αolefin soluble in purified ADMA-16) was detected by GC-MS, and its concentration was about 700 ppm.

The sample that had been stored in a metal alkyl cylinder under nitrogen for 6 months at 22° C. was found to contain 0.03 ppm of TMA, 5 ppm of DMA and approximately 5 ppm of MI. The sample had not developed any malodorous smell and there were no indications of degradation.

The results are reported in Table 2, above.

### Example 9

### Stability of Purified ADMA-12 Under Nitrogen

A sample of the purified ADMA-12 product from Example 6 was used for the aging study of this Example. The purified sample was stored in a glass bottle under nitrogen in a dry-box at 22° C. and 2 ml fractions were taken for analysis in the fourth and again in the fifth month since purification. The 2 ml samples were analyzed for TMA, DMA and N-methylimine. There were no substantial differences in the concentrations of these impurities in the purified and aged, purified sample. The result is reported in Table 2, above. The data indicated that there was no degradation during storage.

What is claimed:

1. A process for reducing malodorous and salt impurities from an impure alkyldimethylamine (ADMA) product comprising:
(a) washing said impure ADMA product with an amount of water to form a water-washed ADMA product; and
(b) purging said water-washed ADMA product with an inert gas while raising and maintaining the temperature of said water-washed ADMA at an elevated temperature and for an amount of time thus forming a purified ADMA product.

2. The process of claim 1 wherein said purified ADMA product comprises at most about 20 ppm of DMA, at most about 2 ppm of TMA and at most about 20 ppm of N-methylimine.

3. The process of claim 1 further comprising filtering said impure ADMA before step (a).

4. The process of claim 1 further comprising filtering said purified ADMA product after step (b).

5. The process of claim 1 wherein said process further comprises allowing or causing the water-washed ADMA product to separate into an organic phase and an aqueous phase after step (a) by a phase cut and recovering the organic phase.

6. The process of claim 1 wherein said amount of water is in the range of from about 5 to about 20% by weight of said impure ADMA product.

7. (canceled)

8. The process of claim 1 wherein said elevated temperature is in the range of from about 60° C. and about 150° C.

9. (canceled)

10. The process of claim 1 wherein said inert gas is selected from the group consisting of nitrogen, helium, neon, argon, and xenon.

11. The process of claim 5 wherein said process further comprises warming said water-washed ADMA after step (a) to about 80° C. with stirring, standing to allow separation of water and ADMA phases, and removing said water phase by a phase cut.

12. The process of claim 1 wherein said impure ADMA product comprises predominantly individual C₈ to C₁₈ alkyldimethylamines, or any combinations thereof.

13. The process of claim 1 wherein said impure ADMA product comprises greater than about 95% by weight of C₁₆-alkyldimethylamine.

14. The process of claim 1 wherein said impure ADMA product comprises greater than about 95% by weight of C₁₂-alkyldimethylamine.

15. The process of claim 1 wherein said impure ADMA product comprises predominantly a combination of C₈-ADMA product and one other ADMA product selected from C₁₀-C₂₀ ADMA products.

16. The process of claim 1 further comprising adding a masking agent to said purified ADMA product.

17. The process of claim 16 wherein said masking agent is selected from of isoamyl acetate, isoamypropionate, limonene, linolool, β-myrcene, β-phenethyl alcohol and Compounds #80412, #46064 offered by Stanley S. Schoenmann, Inc.

18. A process for the purification of an impure ADMA product comprising:
(a) washing said impure ADMA product with an amount of water equal to in the range of from about 10 to about 20 wt % of said impure ADMA to form a water washed ADMA product;
(b) allowing or causing the water-washed ADMA product to separate into an organic phase and an aqueous phase and recovering the organic phase; and;
(c) purging said organic phase with an inert gas while heating said organic phase to an elevated temperature between about 60° C. and about 150° C.; and maintaining said organic phase at said elevated temperature for a specified amount of time thereby producing a purified ADMA product.

19. The process of claim 18 wherein said purified ADMA product comprises at most about 20 ppm of DMA, at most about 2 ppm of TMA and at most about 20 ppm of N-methylimine.

20. The process of claim 19 wherein said inert gas is selected from the group consisting of nitrogen, helium, neon, argon, and xenon.

21. The process of claim 19 wherein said purging is conducted with nitrogen at a flow rate in the range of from 10 to 15 standard cubic feet per hour (“SCFH”).

22. The process of claim 21 wherein said elevated temperature is in the range of from about 100° C. and about 130° C.

23. The process of claim 22 wherein said washing of step (a) comprises stirring said ADMA and said water at about 80° C.

24. The process of claim 22 further comprising filtering said ADMA product before step (a).

25. The process of claim 22 further comprising filtering said ADMA after step (c).

26. A purified ADMA product comprising predominantly C₈ to C₁₆ alkyldimethyamines or combinations thereof, wherein said purified ADMA product comprises less than about 20 ppm of DMA, less than about 2 ppm TMA and less than about 20 ppm of N-methylimine.

27. The purified ADMA product of claim 26 wherein said purified ADMA product comprises less than about 0.1 wt % of residual H₂O.

28. (canceled)

29. The purified ADMA product of claim 26 wherein said purified ADMA product comprises an odor-masking agent.

30. The purified ADMA product of claim 27 wherein said odor masking agent is amyl acetate.

31. The purified ADMA product of claim 26 wherein said purified ADMA product comprises predominantly C₁₆-alkyldimethylamine and a masking agent.

32. The purified ADMA product of claim 26 wherein said purified ADMA product comprises predominantly C₁₂-alkydimethylamine.

33. The purified ADMA product of claim 26 wherein said purified ADMA product comprises predominantly a combination of C₁₄ and C₁₆ alkyldimethylamines.

34. The purified ADMA product of claim 26 wherein said purified ADMA product comprises predominantly a combination of purified C₈ ADMA product and at least one other purified ADMA product from the group consisting of purified C₁₀ to C₂₀ ADMA products.

35. The purified ADMA product of claim 26 wherein said purified ADMA product has no substantial changes in the levels of DMA, TMA and methylamine after stored sealed for no less than about six months under an inert atmosphere.

36. The purified ADMA product of claim 26 wherein said purified ADMA product has no substantial changes in the levels of DMA, TMA and methylamine after stored sealed for no less than about twelve months under an inert atmosphere.

37. A purified ADMA product made by a process comprising:
(a) washing said impure ADMA product with an amount of water to form a water washed ADMA product; and
(b) purging said water-washed ADMA product with an inert gas while raising and maintaining the temperature of said water-washed ADMA at an elevated temperature and for an amount of time thus forming a purified ADMA product.

38. The purified ADMA product of claim 37 wherein said purified ADMA product comprises less than about 20 ppm of DMA, less than about 2 ppm of TMA, less than about 20 ppm of N-methylimine, and less than about 0.1 wt % of water.

39. The purified ADMA product of claim 37 wherein said amount of water is in the range of from about 10 wt % to 20 wt % of said impure ADMA product and said elevated temperature is in the range of from about 100° C. and about 150° C.

40. The purified ADMA product of claim 39 wherein said method further comprising filtering said impure ADMA product before step (a).

41. The purified ADMA product of claim 39 wherein said method further comprising filtering said purified ADMA product after step (b).

42. The purified ADMA product of claim 41 wherein said filtering removes solids, wherein said solids are selected from metal halides, ammonium bromides and amine oxides and any combinations thereof

43. The purified ADMA product of claim 39 wherein said method further comprises allowing or causing the water-washed ADMA product to separate into an organic phase and an aqueous phase after step (a) by a phase cut and recovering the organic phase.

44. The purified ADMA product of claim 43 wherein said water-soluble impurities are selected from metal salts, amine oxides, TMA, DAM, N-N-dimethylformamide, N-methylformide, and any combinations thereof.

45. The purified ADMA product of claim 37 wherein said purified ADMA product further comprises a masking agent, said masking agent is selected from isoamyl acetate, isoamypropionate, limonene, linolool, β-myrcene, β-phenethyl alcohol and Compounds #80412, #46064 offered by Stanley S. Schoenmann, Inc.

46. (canceled)

47. The purified ADMA product of claim 37 wherein said purified ADMA product comprises predominantly individual C₈ to C₁₆ alkyldimethylamine or any combinations thereof.

48. The purified ADMA product of claim 47 wherein said purified ADMA product comprises predominantly C₁₆ alkyldimethylamine.

49. The purified ADMA product of 47 wherein said purified ADMA product comprises predominantly C₁₂ alkyldimethylamine.

50. The purified ADMA product of claim 47 wherein said purified ADMA product comprises predominantly a combination of purified C₈ ADMA product and at least one other purified ADMA product selected from purified C₁₀ to C₂₀ ADMA products.

51. An amine synergist comprising a purified ADMA product according to any of claims 26, 27, 29-37.

52. The use of a purified ADMA product according to any of claims 26, 27, 29 or 31-36 as an amine synergist.

53. A method of synergizing a photoinitiation reaction comprising:
a. combining i) at least one photopolymerizable monomer and/or oligomer, ii) at least one photopolymerization initiator, iii) at least one purified ADMA product, iv) at least one short chain tertiary amino compound containing at least two electronegative atoms; and
b. contacting said photopolymerizable mixture with radiation, thus producing a photopolymerized article.

54. The method according to claim 53 wherein said photopolymerizable mixture is formed by combining in the range of from about 0.5 to about 85wt %, based on the photopolymerizable mixture, of at least one photopolymerizable monomer with the other components ii)-iv).

55. The method according to claim 53 wherein said photopolymerizable mixture is formed by combining in the range of from about 50 to about 70wt %, based on the photopolymerizable mixture, of at least one photopolymerizable monomer with the other components ii)-iv).

56. The method according to claim 54 wherein said photopolymerizable mixture comprises only one photopolymerizable monomer.

57. The method according to claim 56 wherein said photopolymerizable monomer is selected from acrylates, methacrylates, and the like.

58. The method according to claim 54 wherein said at least one photopolymerizable monomer and/or oligomer is a polyfunctional monomer and/or oligomer, wherein said polyfunctional monomers and/or oligomers is characterized as compounds or oligomers having more than one alpha-beta-ethylenic site of unsaturation.

59. The method according to claim 57 wherein alpha, beta-ethylenically unsaturated carboxylic acids are used in conjunction with said acrylate and/or methacrylate monomers.

60. The method according to any of claims 53-55 wherein said photopolymerizable mixture is formed with only one photopolymerizable monomer.

61. The method according to claim 54 wherein said photopolymerizable monomer is selected from tripropylene glycol diacrylate, trimethylol propane tetraacrylate, ethoxylated trimethylol propane tetraacrylate, propoxylated neopentyl glycol diacrylate, hexanediol diacrylate, the like, and mixtures of any two or more thereof.

62. The method according to claim 54 wherein said photopolymerizable mixture is formed by combining in the range of from about 0.01 to about 10 parts by weight, per 100 parts by weight of the at least one photpolymerizable monomer, of said at least one photoinitiator with the other components i), iii) and iv).

63. The method according to claim 61 wherein said photopolymerizable initiators is selected from Type I photoinitiators, Type II photoinitiators, and mixtures thereof.

64. The method according to claim 59 wherein component ii) comprises a Type I photoinitiator.

65. The method according to claim 54 wherein said photopolymerizable mixture is formed by combining in the range of from about 0.1 parts to about 15 parts, based on the weight of total formulation, of the at least one short chain amine with the other components i)-iii).

66. The method according to claim 53 wherein said electronegative atoms of said short chain tertiary amino compound containing at least two electronegative atoms are bonded only to short chain alkyl or alkylene groups, wherein the short chain tertiary amino compound has a total of at least 4 abstractable hydrogen atoms in positions alpha to at least some of the electronegative atoms in the compound.

67. The method according to claim 65 wherein said electronegative atoms of said short chain tertiary amino compound containing at least two electronegative atoms are bonded only to short chain alkyl or alkylene groups, wherein the short chain tertiary amino compound has a total of at least 6 abstractable hydrogen atoms in positions alpha to at least some of the electronegative atoms in the compound.

68. The method according to claim 64 wherein said at least one short chain amino tertiary amino compound is represented by the formula:
R—(CH₂)ₙ—NR¹R²
where
A) R is (i) a dialkylamino group in which each alkyl is, independently, a C₁₋₃ primary alkyl group; (ii) an N-alkylpiperazinyl group in which the alkyl is a C₁₋₃ primary alkyl group, or (iii) a morpholino group; (iv) a C₁₋₃ alkylhydoxy group.
R¹ is an alkylhydoxy group or a dialkylamino group in which each alkyl is, independently, a C₁₋₃ primary alkyl group;
R² is (i) a dialkylamino group in which each alkyl is, independently, a C₁₋₃ primary alkyl group; (ii) an alkyleneamino group in which alkylene is a C₁₋₃ alkylene group and the amino is a dialkylamino group in which each alkyl is, independently, a C₁₋₃ primary alkyl group; (iii) an alkyleneaminoalkyleneamino group (—R—N(R)—R—NR₂) in which each alkylene is, independently, a C₁₋₃ alkylene group, the amino between the alkylenes is a C₁₋₃ primary alkylamino group, and the other amino is a dialkylamino group in which each alkyl is, independently, a C₁₋₃ primary alkyl group; (iv) an alkyleneoxyalkyleneamino group (—R—O—R—NR₂) in which each alkylene is, independently, a C₁₋₃ alkylene group, and the amino is a dialkylamino group in which each alkyl is, independently, a C₁₋₃ primary alkyl group; or (v) an alkyleneoxyalkyleneoxyalkyleneamino group (—R—O—R—O—R—NR₂) in which each alkylene is, independently, a C₁₋₃ alkylene group, and the amino is a dialkylamino group in which each alkyl is, independently, a C₁₋₃ primary alkyl group;
or where
B) R is (i) a dialkylamino group in which each alkyl is, independently, a C₁₋₃ primary alkyl group; (ii) an N-alkylpiperazinyl group in which the alkyl is a C₁₋₃ primary alkyl group, or (iii) a morpholino group; and R¹ and R² taken together is (i) an N-alkylpiperazinyl group in which the alkyl is a C₁₋₃ primary alkyl group, or (ii) a morpholino group.

69. The method according to claim 53 wherein said short chain tertiary amino compound consists of one or more tertiary amino groups, one or more ether oxygen atoms, and/or one or two hydroxyl groups linked to each other by C₁₋₃ alklylene groups, such that there are at least two tertiary amino groups or at least one tertiary amino group and at least one ether oxygen atom or at least one hydroxyl group linked together in this fashion, and such that the compound has a total of at least 4 abstractable hydrogen atoms in positions alpha to at least some of the electronegative atoms in the compound.

70. The method according to claim 53 wherein said purified ADMA product is produced by (a) washing an impure ADMA product with an amount of water to form a water-washed ADMA product; and (b) purging said water-washed ADMA product with an inert gas while raising and maintaining the temperature of said water-washed ADMA at an elevated temperature and for an amount of time thus forming a purified ADMA product.

71. The method according to claim 64 wherein said purified ADMA product comprises at most about 20 ppm of DMA, at most about 2 ppm of TMA and at most about 20 ppm of N-methylimine.

72. The method according to claim 71 wherein said purified ADMA product comprises less than about 0.1 wt % of residual H₂O.

73. The method according to claim 71 wherein said purified ADMA product comprises an odor-masking agent.

74. The method according to 73 wherein said purified ADMA product predominantly at least one of C₁₆-alkyldimethylamine; C₁₂-alkydimethylamine; and a combination of C₁₄ and C₁₆ alkyldimethylamines.

75. The method according to claim 71 wherein said purified ADMA product comprises predominantly a combination of purified C₈ ADMA product and at least one other purified ADMA product selected from purified C₁₀ to C₂₀ ADMA products.

76. The method according to 70 wherein said purified ADMA product has no substantial changes in the levels of DMA, TMA and methylamine after stored sealed for no less than about six months under an inert atmosphere.

77. The method according claim 69 wherein the impure ADMA product contains soluble impurities selected from metal salts, amine oxides, TMA, DAM, N-N-dimethylformamide, N-methylformide, and any combinations thereof.

78. The method according to claim 63 wherein said at least one purified ADMA product and said at least one short chain tertiary amino compound have a synergistic effect on the photopolymerization reaction.

79. The method according to claim 70 wherein at least one of a) pigments and dyes; b) light stabilizers; c) one or more radical scavengers; and d) adhesion promoters is combined with components i)-iv) to form a photpolymerizable compound.

80. The method according to claim 79 wherein the contacting of the photopolymerizable mixture with radiation effects the synergistic photopolymerization of the photopolymerizable monomer thus forming a photopolymerized article.

81. The method according to claim 80 wherein the photopolymerizable mixture is contacted with said radiation when: said photopolymerizable mixture is a thin coating on a traveling web; said photopolymerizable mixture is a coating or laminate on a substrate; or when it is an article or shape in a mold.

82. The method according to claim 81 wherein the photopolymerization is contacted with said radiation under continuous or intermittent conditions.

83. The method according to claim 82 wherein said photopolymerizable mixture is contacted with said radiation when it is a thin coating on a traveling web, wherein said thin coating is a film having a thickness of about 2 mils or less that is formed by a coating system operating at high linear speeds.

84. The method according to claim 83 wherein said thin coating is a thin film used in the manufacture of thinly-coated papers or thin high grade card or paperboard stock used in producing magazine covers, brochures, corporate annual reports, folders, and the like.

85. The method according to claim 83 wherein said thin coating is contacted with said radiation for a period of time ranging from about 0.005 to about 0.02 seconds.

86. The method according to claim 79 wherein said photopolymerized compositions is used as a photopolymerizable ink or coating applied as printed, decorative, or pictorial matter on a substrate.

87. The method according to claim 53 wherein said radiation is selected from coherent or non-coherent radiation.

88. The photopolymerized article formed by the method according to claim 53.
